(19) United States
(12) Patent Application Publication
(10) Pub. No.: US 2009/0261526 A1
(43) Pub. Date: Oct. 22, 2009

(54) FEEDING DEVICE AND FEEDING METHOD

(75) Inventor: Jouko TUOMIKUMPU, Kajaani (FI)

Correspondence Address: OLIFF & BERRIDGE, PLC, P.O. BOX 320850, ALEXANDRIA, VA, 22320-4850, US

(73) Assignee: METSO AUTOMATION OY, HELSINKI (FI)

(21) Appl. No.: 12/420,243

(22) Filed: Apr. 8, 2009

(30) Foreign Application Priority Data
Apr. 17, 2008 (FI) 20085327

(51) Int. Cl. B65H 5/00; G01N 33/34

(52) U.S. Cl. 271/264; 73/159

(57) ABSTRACT

A feeding device comprises at least two feeding units. At least one feeding unit may send a sample towards the measurement device so that the sample passes at least one feeding unit next in the feed direction.

### FIELD

The invention relates to a feeding device and a feeding method for feeding a sample to a measuring apparatus.

### BACKGROUND

In conventional paper, pulp and board industry finished products or those being manufactured are sampled and conveyed to a separate measuring apparatus, i.e. an analyzer, for analysis. The characteristics of a product to be analyzed include formation, fiber alignment, square weight, thickness, roughness, air permeability, glare, colour and moisture, and bursting, tearing and tensile strength. The length of the sample may be equal for example to the width of the web to be manufactured, such as 10 meters, and its width may be 30 cm, for example. The sample may also be taken from the material to be manufactured in machine direction, in which case the sample may be longer still, hundreds of meters even. The sample may also be a standard size A4 sheet, for example.

The measuring apparatus analyzing the samples usually comprises a reception opening through which the samples are fed in series for analysis. Before the analysis the samples are placed to a feeding device that automatically feeds the samples in a desired order to the measuring apparatus.

The feeding device may contain a plural number of superimposed cartridges. The operator places one sample into each cartridge and when doing this, s/he may be standing behind the feeding device or at the side thereof and place the samples to the cartridges, which are on different levels, from the back or the side. The sample may be in the form of a sheet or a roll. The cartridges may be displaced vertically with an elevator-type movement, the cartridge of the first sample to be measured being driven first to the reception opening of the measuring apparatus. When the first sample is to be measured, the feeding device feeds the sample into the measuring apparatus. Next, the following cartridge is driven to the reception opening to wait to be fed for analysis. This is repeated one at a time for all cartridges containing a sample.

However, the prior art solution involves problems. Separate cartridges are needed for feeding the samples. Inserting the samples into the cartridges and handling of the cartridges is slow and inconvenient. The elevator-type moving of the cartridges is also slow, complicated and requires numerous moving parts. In addition, many limit switches, motors and I/O devices are required for controlling the movements of the cartridges and the elevator. Placing of the samples into the cartridges is slow and inconvenient because the samples must be set at different heights, or each cartridge must be moved to a suitable positioning height before a sample is placed into the cartridge. Moreover, it may be difficult to monitor the movement of the samples to the analysis, because it is difficult to see on the different height levels. Also the fact that board and sheet samples deviating from standard dimensions cannot be measured may cause problems.

### SUMMARY

An object of the invention is to provide an improved feeding device and method. This is achieved by a feeding device which is arranged to feed paper and/or board samples to a measuring apparatus for measurement. The feeding device comprises at least two successive feeding units, which are arranged to feed a sample towards measuring to be carried out in the measuring apparatus, the feeding device being arranged to carry a sample sent from at least one feeding unit past at least one feeding unit that is next on the feed path.

The invention further relates to a feeding method for measurement to be carried out in a measuring apparatus for paper and/or board samples. The method comprises the steps of sending a sample by at least one feeding unit of the feeding apparatus towards measurement to be carried out in the measuring apparatus; and guiding the sent sample past at least one feeding unit that is next on the feed path.

Preferred embodiments of the invention are disclosed in the dependent claims.

The method and feeding device of the invention provide a number of advantages. A plural number of samples may be rapidly fed one after the other, and there is little need for moving parts and limit switches. The positioning of the samples is simple and the movement of the samples to the analysis is easy to monitor. In addition, board samples and a sample carrier may be used in the measurement.

### LIST OF FIGURES

In the following the invention will be described in greater detail with reference to preferred embodiments and the accompanying drawings, in which

FIG. 1 is a side view of a feeding device;

FIG. 2 is a diagonal view of the feeding device from above; and

FIG. 3 is a flow diagram of the method.

### DESCRIPTION OF EMBODIMENTS

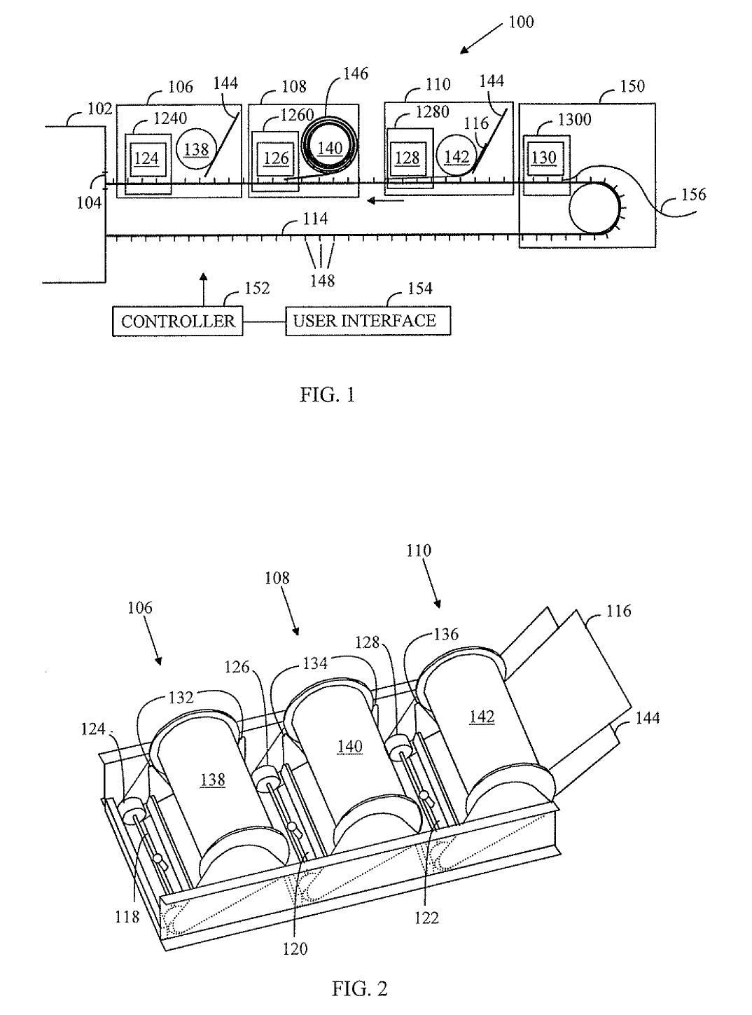

Let us examine first a feeding device with reference to FIG. 1. The feeding device 100 is meant for feeding paper, pulp and/or board samples to analysis that may be carried out in a measuring apparatus 102. The measuring apparatus 102 may measure at least one characteristic of a paper, pulp or board sample that may be used for example for quality control of a finished product or a product that is being manufactured, and for controlling manufacturing operations and further processing.

The feeding device 100 may be placed in front of the measuring apparatus 102. The feeding device 100 comprises at least two successive feeding units 106 to 110, 150, the feeding units 106 to 110, 150 all having the same feeding direction. This means that the feeding units 106 to 110, 150 are one after the other on the feed path. Each feeding unit 106 to 110, 150 may send a sample 116, 146, 156 towards measurement to be carried out in the measurement apparatus 102.

The feeding device 100 may comprise a conveyor 114 for conveying the sample 116, 146, 156 from the feeding unit 106 to 110, 150 to the measuring apparatus 102. In the feeding device 100 the sample 116, 146, 156 is fed onto the conveyor 114 towards the measurement apparatus 102 by at least one feeding unit 108 to 110, 150 of the feeding device 100. The sample 116, 146, 156 carried by the conveyor 114 may travel past at least one other feeding unit 106 to 110 that is on the feed path between the at least one feeding unit 108 to 110, 150 and the measurement apparatus 102.

The conveyor 114, which may comprise one or more conveyor belts, a set of drums, a set of wheels, or the like, one after the other and/or side by side, may convey the sample 116, 146, 156 to a possible reception opening 104 in the measurement apparatus 102 or even into the reception opening 104 (for a distance) for measurement. Every feeding unit 106 to 110 of the feeding device 100 may be provided with a feed switch 1240 to 1280, which is based on a press 124 to 130 and the conveyor 114. When the press 124 to 130 presses the sample 116, 146 against the conveyor 114, the feed switch 1240 to 1280 may be thought of as being closed. The feed switch 1240 to 1280 may be closed when the sample 116, 146 is to start off towards the measuring apparatus 102 carried by the conveyor 114. Otherwise the feed switch 1240 to 1280 may be open, which means that there is a gap between the conveyor 114 and the press 124 to 130. In that case the sample 116, 146 carried by the conveyor 114 may travel through a feed switch 1240 to 1280 provided in connection with a feeding unit 106 to 110 located next on the feed path. The feed switches 1240 to 1280 may be closed and opened with a controller 152. Although not shown in FIG. 1, a similar switch is also conceivable in connection with the feeding device 150.

In the feeding device 100 the sample 116, 146, 156 may be pushed into the feeding unit 106 to 110, 150 in such a way that the sample 116, 146, 156 penetrates underneath the press 124 to 130. This may be carried out manually by the operator, or the feeding unit 106 to 110, 150 may pull the sample 116, 146, 156 placed into the feeding unit for a suitable length ready for insertion into the feeding apparatus. Each feeding unit 106 to 110, 150 is designed in such a way that a gap may be left between the sample 116, 146, 156 and the conveyor 114, if the press 124 to 130 has not been arranged to press the sample 116, 146, 156 against the conveyor 114. In that case the feed switch 1240 to 1280 may be thought of as being open. The feeding device 100 thus keeps the sample 116, 146, 156 and the conveyor 114 physically apart from one another, unless the press 124 to 130 has been guided or arranged to press the sample 116, 146,156 against the conveyor 114. If there is no sample 116, 146, 156 in the feeding unit 106 to 110, 150, the press 124 to 130 may be kept apart from the conveyor 114. This allows a sample 116, 146, 156 from a feeding unit farther behind to be conveyed to the measurement apparatus 102 past a feeding unit ahead.

When a sample 116, 146, 156 located in the desired feeding unit 106 to 110, 150 is to be transferred to measurement, the press 124 to 130 of the feeding unit 106 to 110, 150 may be set to press the sample 116, 146, 156 against the conveyor 114. The friction between the conveyor 114 and the sample 116, 146, 156 pulls the sample 116, 146, 156 along towards the reception opening 104. In addition or alternatively, the conveyor 114 (the conveyor belt) may be provided with protrusions 148 that press into the sample 116, 146, 156, thus reinforcing the conveyor's 114 hold of the sample 116, 146, 156. The conveyor belt 114 with protrusions may be a spiked conveyor belt, for example.

Each feeding unit 106 to 110, 150 may comprise at least one limiter 118, 120, 122, which may prevent the sample 116, 146 from being drawn away from under the press 124 to 130 when the feeding unit 106 to 110 is not feeding a sample to the conveyor 114. This ensures that a sample 116, 146 placed into each feeding unit 106 to 110 may be pressed with the press 124 to 130 against the conveyor 114 and fed to measurement at a desired moment. Although not shown in the Figures, also the feeding unit 150 may comprise a limiter.

At least one feeding unit 106 to 110 may comprise a holder 132, 134, 136 for a cylinder 138 to 142. The rigid cylinder 138 to 142, which may be made of metal or plastic, for example, serves as a support in the centre of a roll-shaped sample 146. The at least one feeding unit 106 to 110 may feed the sample 146 rolled onto the cylinder 138 to 142 to the conveyor 114 for conveyance towards measurement.

At least one feeding unit 106 to 110 may comprise a sample support 144 for a sheet-like sample 116. The sample support 144 supports the sheet-like sample 116, thus allowing it to be easily fed with the feeding unit 106 to 110 onto the conveyor 114 towards measurement. The sample support 144 may be a metal or plastic plate, for example, attached to the feeding unit.

The feeding device 100 may be operated so that the operator placing a sample 116, 146 into each feeding unit 106 to 110 is at the side of all feeding units 106 to 110. The height of the feeding unit 106 to 110 may be adjusted according to the operator and hence the feeding units 106 to 110 may be at a height of 90 cm from the floor, for example. This is an ergonomic improvement.

The feeding device 100 allows samples to be fed automatically to the measurement apparatus 102 for analysis. Automated feeding and analysis of a plural number of samples may take dozens of minutes. Different samples may be prioritized and the samples fed in their order of priority for measurement.

If in the middle of an ongoing measurement process a new sample is to be analyzed, ongoing sample transfer may be interrupted. The feeding device 100 may interrupt a predetermined sample dispatch process and feed an interrupting sample 116,146 into the measurement apparatus 102 for example if a sudden need arises to quickly analyze a new sample. After the interruption the predetermined sample dispatch process may be resumed from where it was left off before the interruption. The interruption may be accomplished by the operator, who issues an interrupt command through a user interface 154. When the interrupting sample or samples have been fed into the measurement apparatus 102, the interrupted feed program may be resumed automatically or by the operator's command.

The open roll representing the sample 156 may be fed for analysis from the end of the feeding device 100 and thus the end of the feeding device 100 may also act as a feeding unit 150. The open roll may be a long sample taken in the machine direction and wound into a loose roll without a rigid centre cylinder as a support. The sample may be of paper or board.

Non-standard sized sheet samples and particularly those smaller than the standard size may be fed with the help of a separate sample carrier (not shown in the figures) whereto the sheet sample 116 may be attached. The sample carrier is dimensioned so that it may be fed to the analysis similarly as the open roll 156 from the end of the feeding device 100 serving as one feeding unit 150. The open roll 156 and the sample carrier may be fed into the analysis with the feeding device 100. Both the open roll 156 and the sample carrier are pushed between the press 130 and the conveyor 114 to be ready for feeding into the measurement apparatus 102. The press 130 and the conveyor 114 form a feed switch 1300 that operates similarly as the feed switches 1240 to 1280.

In connection with the feeding of both the open roll and the sample carrier the feeding units 106 to 110 may be passed by maintaining the feed switches 1240 to 1280 open.

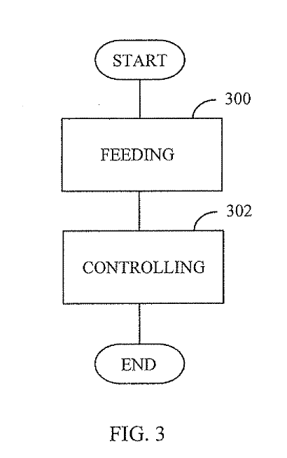

FIG. 3 is a flow diagram of the method. In step 300 a sample 116, 146 is sent with at least one feeding unit 106 to 110, 150 of the feeding device 100 towards measurement to be carried out in the measurement apparatus 102. In step 302 the sent sample 116, 146 is guided past at least one feeding unit 106 to 110 next on the feed path, the feeding unit being between the at least one feeding unit 108 to 110 and the measurement apparatus 102 in the feed direction.

The controller 152, which may comprise a processor, memory and an appropriate computer program or programs, may in turn control the operation of the feeding device 100, which includes the steps shown in the flow diagram. In addition, through the user interface 154 operationally connected to the controller 152 the user may act on at least some of the operations of the controller 152, or directly control the feeding device 100 manually. The user may interrupt the feed program and feed an interrupting sample or samples into the measurement apparatus through an appropriate feeding unit or feeding units.

Although the invention is disclosed above with reference to the examples of the accompanying drawings, it is obvious that the invention is not restricted to these but may be varied in many ways within the scope of the accompanying claims.

1. A feeding device configured to feed paper and/or board samples for measurement in a measurement apparatus, the feeding device comprising at least two successive feeding units configured to send a sample towards measurement to be carried out in the measurement apparatus, and the feeding device being configured to carry a sample sent from at least one feeding unit past at least one feeding unit next on the feeding path.

2. The feeding device as claimed in claim 1, wherein the feeding device comprises a conveyor for carrying a sample from each feeding unit to the measurement apparatus.

3. The feeding device as claimed in claim 2, wherein the feeding device comprises a press in connection with each feeding unit; and
the press is arranged to press a sample against the conveyor for feeding the sample for measurement.

4. The feeding device as claimed in claim 3, wherein the at least one feeding unit comprises a limiter arranged to prevent a sample from being drawn away from under the press when the sample is in a feeding unit that is not sending the sample for measurement.

5. The feeding device as claimed in claim 3, wherein in each feeding unit that is not sending a sample for measurement the feeding device is arranged to hold the sample and the conveyor physically apart in order to allow the sample to be carried past, and the feeding device is arranged to press the sample against the conveyor with the press, when the feeding device is sending the sample for measurement.

6. The feeding device as claimed in claim 1, wherein the at least one feeding unit comprises a holder for a sample roll; and the at least one feeding unit is arranged to send a sample on the roll for measurement.

7. The feeding device as claimed in claim 1, wherein the at least one feeding unit comprises a sample support for a sheet-like sample.

8. The feeding device as claimed in claim 1, wherein the feeding device is arranged to interrupt a predetermined sample dispatch process, send an interrupting sample for measurement and resume the predetermined sample dispatch process after the interruption.

9. The feeding device as claimed in claim 1, wherein the feeding device is arranged to receive a sample from an open roll.

10. The feeding device as claimed in claim 1, wherein the feeding device is arranged to receive a sample carrier whereto the sample has been attached.

11. A feeding method for a measurement of paper and/or board samples in a measurement apparatus, the method comprising:
sending a sample with at least one feeding unit of a feeding device towards measurement to be carried out in the measurement apparatus; and
guiding the sent sample past at least one feeding unit next on the feeding path.

12. The method as claimed in claim 11, the method further comprising pressing the sample with a press against a conveyor and carrying the sample on the conveyor so as to feed the sample for measurement.

13. The method as claimed in claim 11, the method further comprising using at least one limiter for preventing the sample from being drawn away from underneath the press in a feeding unit that is not sending a sample for measurement.

14. The method as claimed in claim 11, the method further comprising keeping the press apart from the conveyor, whereby the sample of the feeding unit corresponding to the press remains physically apart from the conveyor and does not proceed to measurement so as to allow it to be carried past, and pressing the sample against the conveyor with the press when the sample is being sent for measurement.

15. The method as claimed in claim 11, the method further comprising sending a sample in a roll through at least one feeding unit to the measurement apparatus.

16. The method as claimed in claim 11, the method further comprising sending a sheet-like sample through at least one feeding unit to the measurement apparatus.

17. The method as claimed in claim 11, the method further comprising interrupting a predetermined sample dispatch process, sending an interrupting sample to the measurement apparatus and resuming the predetermined sample dispatch process after the interruption.

18. The method as claimed in claim 11, the method further comprising sending a sample from an open roll through a separate feeding unit.

19. The method as claimed in claim 11, the method further comprising sending a sample carrier whereto the sample is attached for measurement.
